(19) United States
(12) Patent Application Publication
(10) Pub. No.: US 2026/0155226 A1
(43) Pub. Date: Jun. 4, 2026

(54) SYSTEM AND METHOD FOR GENERATING ANTICANCER DRUG CANDIDATE COMPOUND BASED ON GENOTYPE

(71) Applicant: Gwangju Institute of Science and Technology, Gwangju (KR)

(72) Inventors: Hojung NAM, Gwangju (KR); Hyunho KIM, Gwangju (KR)

(73) Assignee: Gwangju Institute of Science and Technology, Gwangju (KR)

(21) Appl. No.: 19/358,662

(22) Filed: Oct. 15, 2025

(30) Foreign Application Priority Data
Dec. 4, 2024 (KR) 10-2024-0178434

(51) Int. Cl. G16H 20/10; G06N 20/00; G16B 20/10; G16B 20/20; G16B 40/20; G16H 50/70

(52) U.S. Cl. CPC G16H 20/10; G06N 20/00; G16B 20/10; G16B 20/20; G16B 40/20; G16H 50/70

(57) ABSTRACT

Disclosed herein is a system for generating an anticancer drug candidate compound based on genotype. The system for generating an anticancer drug candidate compound based on genotype of the present invention includes: a processor configured to determine compound information corresponding to a generation condition including genotype information; and a memory, wherein the memory includes: a first encoder configured to determine an encoding vector corresponding to the generation condition; a diffusion model configured to determine a representation vector generated from the encoding vector; and a decoder configured to determine compound information by decoding the generated representation vector.

### CROSS REFERENCE TO RELATED APPLICATION

The present application claims priority to Korean Patent Application No. 10-2024-0178434, filed on Dec. 4, 2024, the entire contents of which are hereby incorporated by reference in its entirety.

### BACKGROUND OF THE INVENTION

### Field of the Invention

The present invention relates to a system and method for generating an anticancer drug candidate compound based on a genotype.

### Description of the Related Art

In the conventional new drug development, a large amount of effort, time, and cost is required. It is known that new drug development in a traditional manner requires, on average, 15 years or more and up to 2 trillion won in cost.

Recently, with the advancement of artificial intelligence technology, research and development on various artificial intelligence models for new drug development is being actively conducted in order to reduce the cost of new drug development and increase efficiency.

In particular, de novo drug design refers to a method of proposing a new compound that satisfies a target condition without a base scaffold form. Since 2017, research on de novo drug design using generative artificial intelligence has been actively carried out.

However, most of the research has focused on generating molecular structures that activate or inhibit the activity of a target protein related to the cause or treatment of a disease, by designating the target protein.

However, in the case of complex diseases such as cancer, due to heterogeneity, there is difficulty in specifying target proteins, and thus, there is difficulty in applying the conventional method.

### SUMMARY OF THE INVENTION

The technical object to be solved by the present invention is to provide a system and method for generating an anticancer drug candidate compound based on genotype that generates an effective anticancer drug candidate compound in a de novo manner based on genotype of cancer.

To solve the aforementioned technical object, there is provided a system for generating an anticancer drug candidate compound based on genotype, according to an embodiment of the present invention. The system may include: a processor configured to determine compound information corresponding to a generation condition including genotype information; and a memory, in which the memory may include: a first encoder configured to determine an encoding vector corresponding to the generation condition; a diffusion model configured to determine a representation vector generated from the encoding vector; a decoder configured to determine the compound information by decoding the generated representation vector, and the diffusion model may be a conditional diffusion model configured to determine the generated representation vector by removing noise from random noise based on the encoding vector.

In an embodiment of the present invention, the system may further include: a second encoder trained with parameters associated with the decoder that encodes a compound to determine an embedding vector, in which the first encoder may be trained such that a distance relationship between the encoding vector and the embedding vector by the second encoder is trained, the training may be performed using pair information of already known genotypes and drugs known to be responsive to the corresponding genotypes as training data, and the training method may be performed by contrastive learning.

In an embodiment of the present invention, the first encoder may include: a first module configured to generate an embedding vector from the genotype information; and a second module configured to generate the encoding vector corresponding to the generation condition by performing transformer operation on the embedding vector.

In an embodiment of the present invention, the second module may include a plurality of transformer blocks configured to compute association relationships for different genotypes, and a first transformer block among the plurality of transformer blocks may be configured such that the association computation is limited to computation for adjacent genes.

In an embodiment of the present invention, the genotype may include a partial genomic information of entire human genomic information, and the partial genomic information may be selected from among genomic information related to clinically known cancers.

In an embodiment of the present invention, the genotype may include partial genomic information of entire human genomic information, and each genomic information may be associated with mutation presence information, the mutation presence information including base sequence mutation (MUT, Mutation), copy number amplification (CNA), and copy number deletion (CND).

To solve the aforementioned technical object, there is a method of generating an anticancer drug candidate compound based on genotype, according to an embodiment of the present invention. The method may be performed by at least one processor of a computing device, the method including: inputting a generation condition including genotype information; determining an encoding vector corresponding to the generation condition; determining a representation vector generated from the encoding vector; and determining compound information by decoding the generated representation vector, in which the generated representation vector may be generated by a conditional diffusion model that removes noise from random noise based on the encoding vector.

The present invention has an effect of being capable of generating a new anticancer drug candidate compound that is different from a known chemical structure based on a target protein.

The present invention has an effect of having clinical scalability by using a genotype closely associated with clinical practice.

### BRIEF DESCRIPTION OF THE DRAWINGS

FIG. 1 illustrates a system for generating an anticancer drug candidate compound based on genotype according to an embodiment of the present invention.

FIG. 2 illustrates in detail a partial configuration of the system for generating an anticancer drug candidate compound based on genotype according to an embodiment of the present invention.

FIG. 3 illustrates in detail a partial configuration of the system for generating an anticancer drug candidate compound based on genotype according to an embodiment of the present invention.

FIG. 4 illustrates a method of generating an anticancer drug candidate compound based on genotype according to an embodiment of the present invention.

FIG. 5 illustrates in detail a partial configuration of the method of generating an anticancer drug candidate compound based on genotype according to an embodiment of the present invention.

FIG. 6 schematically illustrates constituent elements related to a first training stage.

FIG. 7 schematically illustrates an information flow in a first encoder and a decoder.

FIG. 8 illustrates in detail a partial configuration of the method of generating an anticancer drug candidate compound based on genotype according to an embodiment of the present invention.

FIG. 9 illustrates in detail a partial configuration of the method of generating an anticancer drug candidate compound based on genotype according to an embodiment of the present invention.

FIGS. 10 and 11 schematically illustrate detailed configurations and operation flows of a second encoder.

FIG. 12 illustrates attention masking applied to a first transformer block.

FIG. 13 schematically illustrates constituent elements related to a second training stage.

FIG. 14 schematically illustrates constituent elements related to a third training stage.

FIG. 15 illustrates in detail a partial configuration of the method of generating an anticancer drug candidate compound based on genotype according to an embodiment of the present invention.

FIG. 16 illustrates a partial stage of the method of generating an anticancer drug candidate compound based on genotype together with related constituent elements according to an embodiment of the present invention.

FIG. 17 is a block diagram illustrating an embodiment of a computing system in which the present invention can be implemented.

FIGS. 18 and 19 are block diagrams illustrating an embodiment of a computing device according to the present invention.

### DETAILED DESCRIPTION OF THE INVENTION

The present invention may be variously modified and may have various embodiments, and particular embodiments illustrated in the drawings will be described in detail below. However, the description of the exemplary embodiments is not intended to limit the present invention to the particular exemplary embodiments, but it should be understood that the present invention is to cover all modifications, equivalents and alternatives falling within the spirit and technical scope of the present invention.

In the description of the present invention, the specific descriptions of publicly known related technologies will be omitted when it is determined that the specific descriptions may obscure the subject matter of the present invention.

Hereinafter, the embodiments of the present invention will be described in detail with reference to the accompanying drawings.

Prior to describing the present invention, a text-to-image generator for generating images from text among conventional artificial intelligence models will be mentioned. The text-to-image generator is a model that generates an image from a string by learning a relationship between features of the string and features of the image. In comparison with this, the present invention uses genotype and responsiveness information instead of text, and uses compound information instead of image. Specifically, the present invention is intended to generate a compound candidate suitable for a specific genotype and responsiveness, by learning a relationship between characteristics relating to genotype and responsiveness information and characteristics relating to compound information.

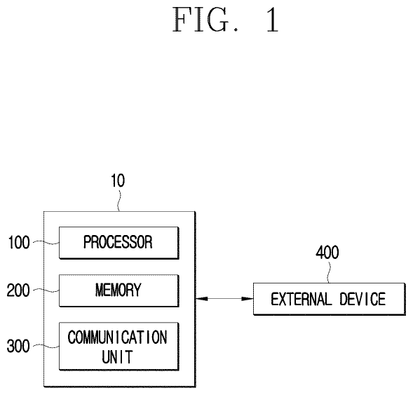

FIG. 1 illustrates a system 10 for generating an anticancer drug candidate compound based on genotype according to an embodiment of the present invention.

With reference to FIG. 1, the system 10 for generating an anticancer drug candidate compound based on genotype according to an embodiment of the present invention includes a processor 100, a memory 200, and a communication unit 300.

The processor 100 is connected to the memory 200 and the communication unit 300, and collects information and controls them.

The processor 100 may be configured as a single physical entity, but may also be configured as a plurality of entities. The processor 100 configured with a plurality of entities may process by dividing a single execution element or may process by separating a plurality of execution elements.

The processor 100 may include at least one of a central processing unit (CPU), a graphic processing unit (GPU), a microprocessor, or an artificial intelligence dedicated processor, and the type of the processor is not limited thereto as long as it performs the functions of the present invention.

The memory 200 may store a program, which is a set of data and executable instructions that may be read or written by the processor 100.

The memory 200 includes storage of non-volatile nature, which may retain data (information) regardless of power supply, and memory of volatile nature, into which data is loaded for processing by the processor and which cannot retain data unless power is provided. The storage may include a flash memory, a hard-disc drive (HDD), a solid-state drive (SSD), or a read only memory (ROM), and the memory may include a buffer, a random access memory (RAM), or a cache.

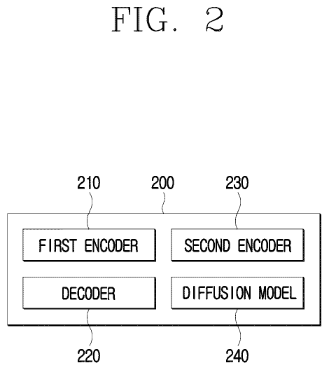

FIG. 2 illustrates in detail the memory 200.

With reference to FIG. 2, the memory 200 includes a first encoder 210, a decoder 220, a second encoder 230, and a diffusion model 240.

The first encoder 210 generates an embedding vector into a latent space from input information. The first encoder 210 corresponds to an encoder of a variational autoencoder (VAE).

The input information to the first encoder 210 may be information on a compound. The information on a compound may be converted into simplified molecular input line entry system (SMILES) information and input.

The decoder 220 generates output information from a representation vector processed in the dimension of the latent space. The decoder 220 corresponds to a decoder 220 of a variational autoencoder (VAE).

The output information is information on a compound, and may be information converted into SMILES information from the generated representation vector. Since the SMILES information is matched to a specific compound, compound information may be acquired from the SMILES information.

The second encoder 230 generates another embedding vector projected into the latent space from genotype information and responsiveness information. The embedding vector generated by the second encoder 230 has the same dimension as the embedding vector generated by the first encoder 210.

The second encoder 230 may be understood as generating a vector related to a drug generation condition, and may be referred to as a condition encoder. Here, the condition may refer to a drug generation condition including genotype and responsiveness information.

The diffusion model 240 performs denoising based on the embedding vector (representation of the generation condition) determined from the second encoder 230, and outputs a generated representation vector.

The generated representation vector is information that may be output as information on a compound by the decoder 220.

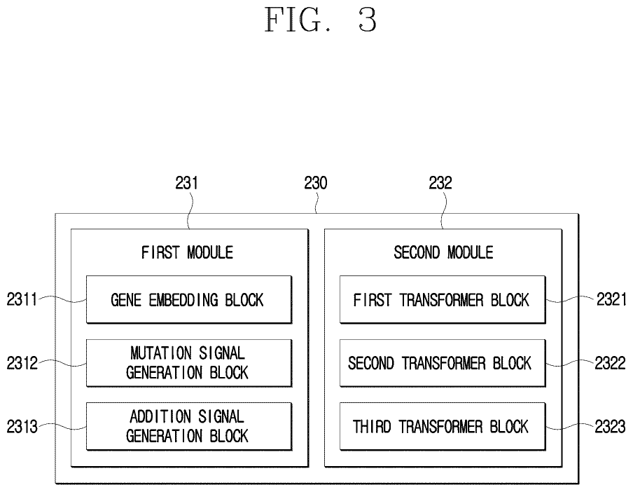

FIG. 3 illustrates the second encoder 230 in detail.

The second encoder 230 includes a first module 231 and a second module 232.

The first module 231 generates an embedding vector related to genotype and responsiveness conditions.

The second module 232 generates a generation condition vector from the embedding vector generated by the first module 231.

The first module 231 includes a gene embedding block 2311, a mutation signal generation block 2312, and an addition signal generation block 2313.

The gene embedding block 2311 generates a genotype embedding vector from genotype information.

The mutation signal generation block 2312 generates a mutation signal vector from mutation information.

The addition signal generation block 2313 generates an addition embedding vector by adding the genotype embedding vector and the mutation signal vector.

The second module 232 includes a first transformer block 2321, a second transformer block 2322, and a third transformer block 2323.

The first to third transformer blocks 2321, 2322, and 2323 compute association relationships for different genotypes. This process may be referred to as a self-attention mechanism. However, the first transformer block 2321 may apply attention masking to limit the computation to adjacent genes.

With reference back to FIG. 2, the communication unit 300 may transmit and receive information with an external device 400 under control of the processor 100.

The communication unit 300 may communicate using at least one method among wired/wireless LAN, Wi-Fi (wireless fidelity), Bluetooth, Zigbee, infrared communication (IrDA, infrared Data Association), near field communication (NFC), wireless broadband internet (WiBro), shared wireless access protocol (SQAP), and RF communication, but the communication method is not necessarily limited to the above-described embodiment.

The external device 400 may refer to any type of device that transmits and receives information with the system 10.

For example, the external device 400 may transmit information requesting inference, along with provision of genotype and responsiveness condition information, to the system 10.

For example, the external device 400 may be an external server that provides training data that may be used for training.

For example, the external device 400 may transmit a control command to the system 10 to control partial operations of the system 10.

For example, the external device 400 may be any one of a server, a smartphone, a tablet PC, a desktop PC, or a notebook, but is not limited to these examples.

Hereinafter, a method of generating an anticancer drug candidate compound based on genotype will be described in detail by way of example as being performed by the system 10 for generating an anticancer drug candidate compound based on genotype.

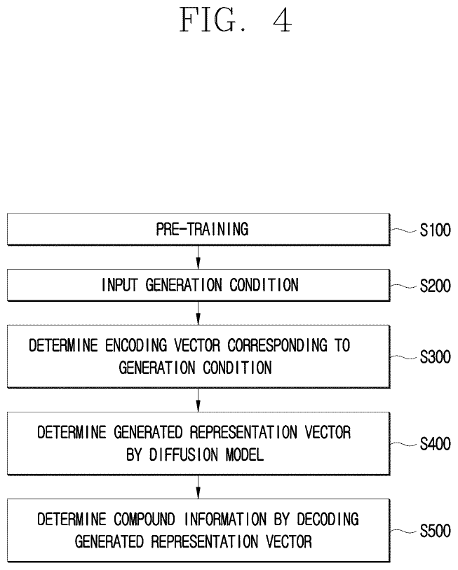

FIG. 4 illustrates a method of generating an anticancer drug candidate compound based on genotype according to an embodiment of the present invention.

With reference to FIG. 4, in step S100, the system 10 performs pre-training. The pre-training may be performed for a plurality of components.

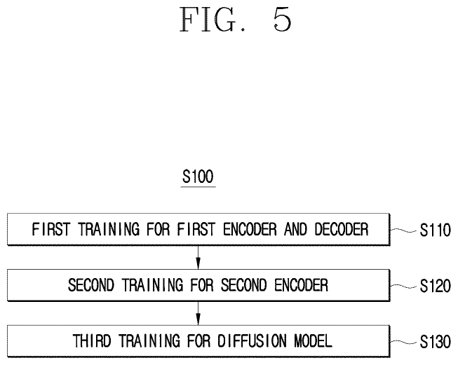

FIG. 5 illustrates step S100 in detail.

With reference to FIG. 5, in step S110, the system 10 performs first training for the first encoder 210 and the decoder 220.

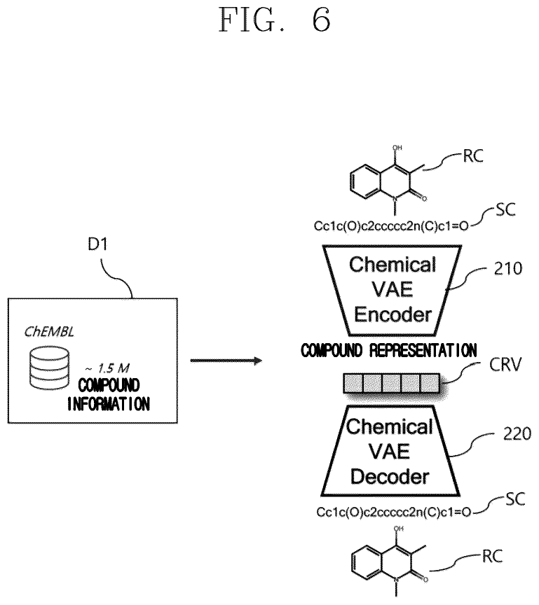

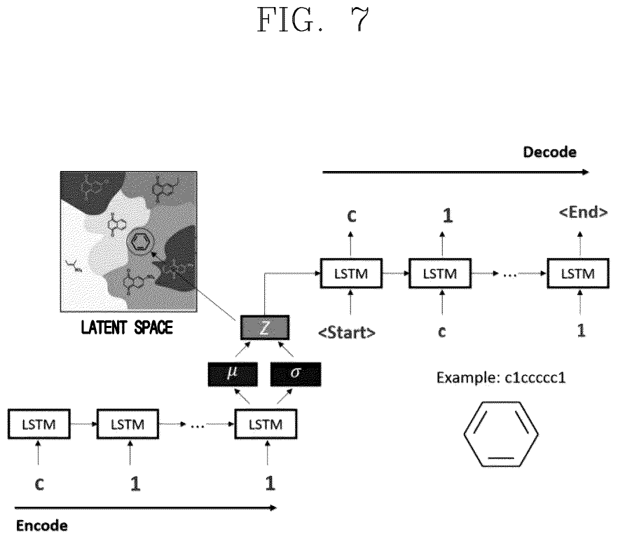

FIG. 6 schematically illustrates components related to the first training stage, and FIG. 7 schematically illustrates information flow in the first encoder 210 and the decoder 220.

The first encoder 210 and the decoder 220 include a long short-term memory (LSTM) structure.

In order to train the transformation of the first encoder 210 and the decoder 220, a real compound RC is converted into a string SC following SMILES. The SMILES string SC may be input to the first encoder 210 or output from the decoder 220.

The first encoder 210 forms a vector Z projected into the latent space from the SMILES string SC. The projected vector may be understood as a compound representation CRV in the latent space.

The compound representation CRV may be input to the decoder 220 and output as the SMILES string SC.

The training of the first encoder 210 and the decoder 220 uses first training data D1. The first training data D1 may be set from approximately 1.5 million pieces of compound information provided from the CheMBL database.

By training with real compounds, the decoder 220 may become a module that has sufficient possibility to extract valid compound information from an arbitrary representation vector CRV. Here, validity may be the possibility of actual physical and chemical existence of the generated compound.

In step S120, the system 10 performs second training for the second encoder 230.

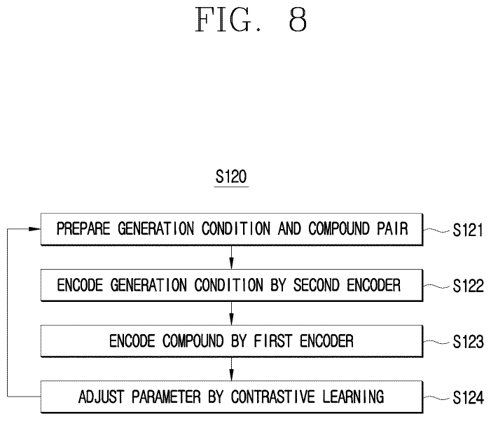

FIG. 8 illustrates step S120 in detail.

With reference to FIG. 8, in step S121, the system 10 prepares a generation condition and compound pair.

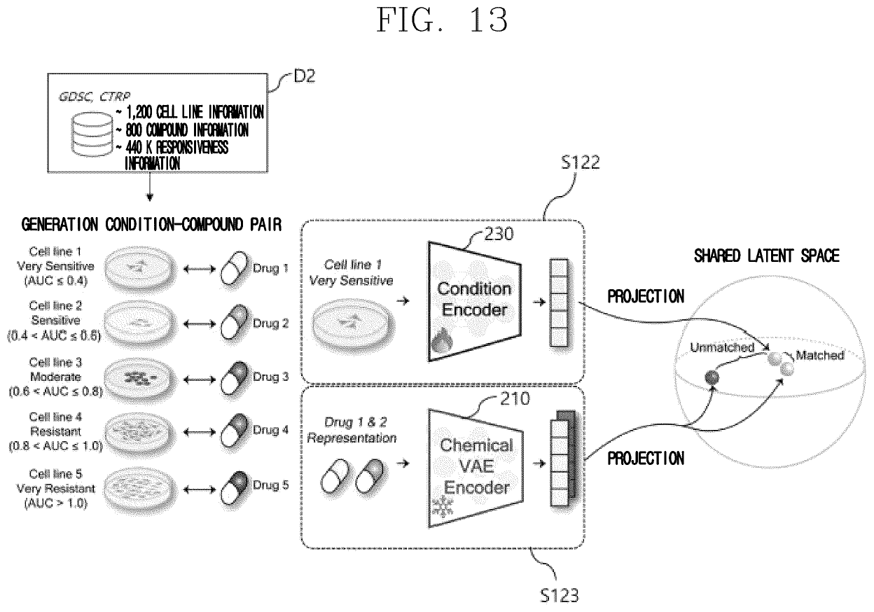

With reference to FIG. 13, in the second training, the second training data D2 may include approximately 1,200 pieces of cell line information, approximately 800 pieces of compound information, and approximately 440,000 pieces of responsiveness information, provided from the GDSC and CTRP databases. Here, the cell line information corresponds to the genotype information.

The GDSC and CTRP databases provide information on responsiveness between each cell line and a specific drug. Drug information may correspond to one compound information.

The responsiveness is classified, according to the AUC value, into very sensitive (AUC≤0.4), sensitive (0.4<AUC≤0.6), moderate (0.6<AUC≤0.8), resistant (0.8<AUC≤1.0), and very resistant (AUC>1.0). In case of the genotype of cancer cells, very sensitive means that the anticancer effect is good.

The generation condition and compound pair means that a genotype and responsiveness are associated with specific compound information. Such an association is based on cell lines and responsiveness information verified in the GDSC and CTRP databases.

For example, a cell line 1 having a first genotype is very sensitive to a first compound (Drug 1). As training data, it is set as the first compound associated with the first genotype and the very sensitive information.

The first encoder 210 generates an embedding vector to be projected into a shared latent space from compound information. The first encoder 210 includes first training parameters determined by the first training stage, and the first training parameters are not adjusted (frozen) in the second training.

In step S122, the second encoder 230 generates an embedding vector related to the generation condition, having the same dimension as the embedding vector, from genotype and responsiveness condition information. The detailed steps are as follows.

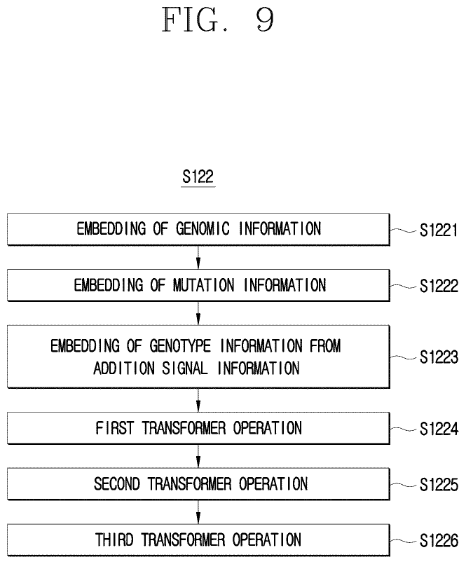

FIG. 9 illustrates step S122 in detail.

With reference to FIG. 9, in step S1221, the gene embedding block 2311 embeds genotype information and generates a first embedding vector.

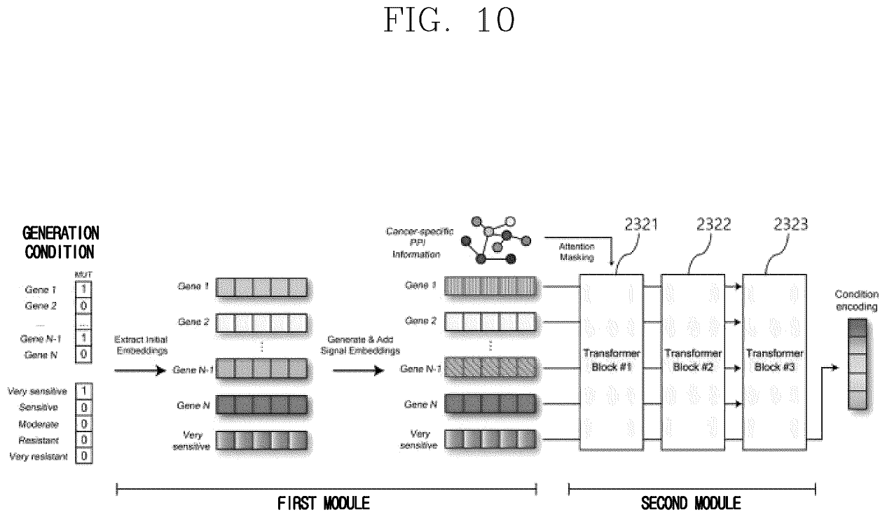

In FIG. 10, genomic information is represented as gene 1 to gene N. Gene 1 to gene N may relate to a portion selected from among human genes. In an embodiment of the present invention, N may be selected as 700. Each gene may be one of 700 pieces of genomic information related to cancers that have been clinically identified to date.

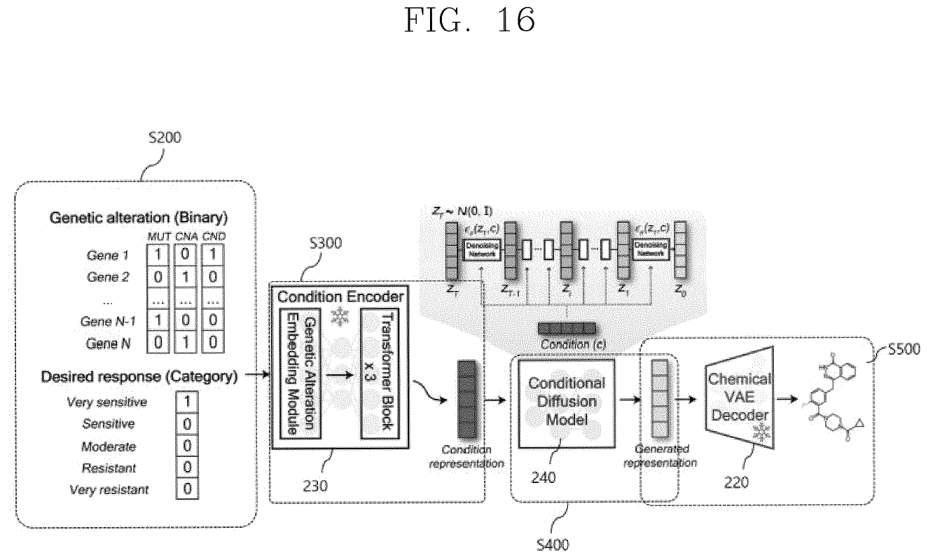

Each genotype may be represented as binary information according to three elements. The elements determining the genotype are base sequence mutation (MUT, Mutation), copy number amplification (CNA), and copy number deletion (CND). Although all of these elements are illustrated in FIG. 16, only base sequence mutation (MUT) is illustrated in FIG. 10 for convenience of explanation.

The leftmost portion of FIG. 10 relates to the generation condition, and represents each of genes and mutation information, and responsiveness information. The right side represents a first embedding vector in which each gene is embedded, using five blocks of different colors. The responsiveness information is also represented as a vector of the same dimension (five blocks).

In step S1222, the mutation signal generation block 2312 embeds mutation information and generates a second embedding vector.

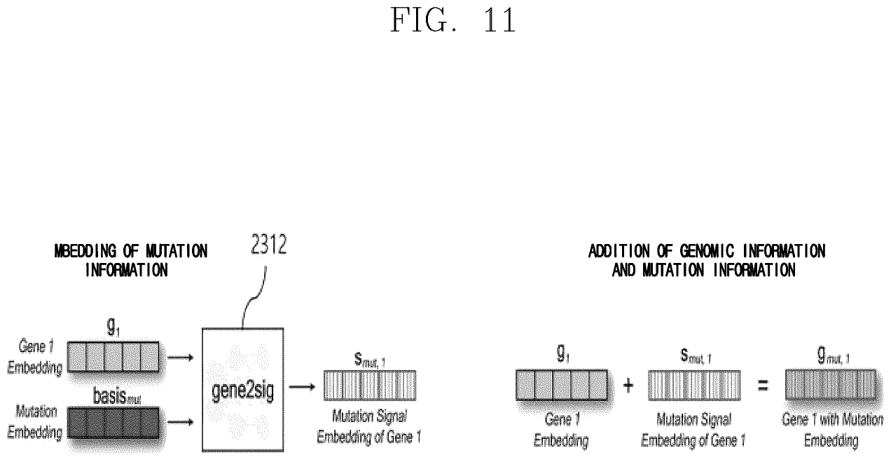

FIG. 11 illustrates embedding of mutation information (left side) and generation of genotype information by adding genomic information and mutation information (right side).

The mutation signal generation block 2312 generates a second embedding vector representing a mutation signal from genomic information and mutation information.

In step S1223, the addition signal generation block 2313 adds the first embedding vector representing genomic information and the second embedding vector representing the mutation signal, and outputs a third embedding vector.

In FIG. 10, information in which the mutation signal (hatching) is added to the genes represented in different colors is illustrated. For the first gene (Gene 1) and the (N-1)th gene (Gene N-1), addition of mutation signal is represented respectively by hatching. The first to Nth genes (Gene 1 to Gene N) may be represented by the first embedding vector or the third embedding vector depending on the presence or absence of mutation. The first embedding vectors or third embedding vectors correspond to the first to Nth genes (Gene 1 to Gene N) correspond to information on the genotype.

The responsiveness information may also be embedded into a vector of the same dimension as the first to third embedding vectors.

The information on the genotype and the responsiveness information embedded by the first module 231 are input to the second module 232.

In step S1224, the first transformer block 2321 performs a first transformer operation.

The first transformer operation computes association relationships among different genotypes. However, unlike the second and third transformer operations, the first transformer operation applies attention masking such that computation of the association relationships is limited to adjacent genes.

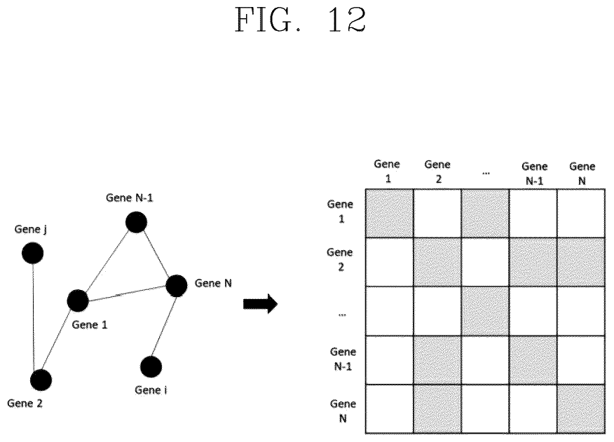

FIG. 12 illustrates adjacency among several genes (left side) and a matrix form representation of the attention masking (right side).

For example, the first gene (Gene 1) has adjacency with the second gene (Gene 2), the (N-1)th gene (Gene N-1), and the Nth gene (Gene N).

The information on adjacency may follow cancer-specific protein-protein interaction (PPI) information.

In the attention masking matrix, portions other than gene pairs that are adjacent to each other are shaded. This means that these gene pairs are not involved in computation of association in the operation.

In step S1225, the second transformer block 2322 performs a second transformer operation.

The second transformer operation computes association relationships among different genotypes.

In step S1226, the third transformer block 2323 performs a third transformer operation.

The third transformer operation computes association relationships among different genotypes.

The second and third transformer operations do not impose limitations on adjacent genes in computing the association relationships. When the distance is two or more, the result is not significantly different from a random association.

The vector related to responsiveness information is output along the first transformer block 2321 to the third transformer block 2323, and the output of the third transformer block 2323 corresponds to a fourth embedding vector, which is the generation condition.

With reference to FIG. 13, in step S122, the second encoder 230 outputs the fourth embedding vector. In addition, in step S123, the first encoder 210 outputs a fifth embedding vector in which compound information is embedded.

The fourth embedding vector and the fifth embedding vector are projected into a shared latent space, and the distance between them may be computed.

In step S124, the system 10 performs the second training for the second encoder 230 by way of adjusting the parameters of modules related to the second encoder 230 in such a way that the distance between the fourth embedding vector and the fifth embedding vector becomes closer when they are associated (matched), and farther when they are not associated (unmatched). This method corresponds to contrastive learning.

FIG. 13 illustrates the projection of a fourth embedding vector by the second encoder 230 for cell line 1 and responsiveness condition 1, and two fifth embedding vectors by the first encoder 210 for drug 1 and drug 2. When the training is well performed, the fourth embedding vector for cell line 1 and responsiveness condition 1 (projection is represented by a pink sphere) and the first drug, which was paired (projection is represented by a yellow sphere), are located close to each other in the shared latent space, while the fourth embedding vector for cell line 1 and responsiveness condition 1 (projection is represented by a pink sphere) and the third drug, which was not paired (projection is represented by a green sphere), is located far from each other in the shared latent space.

By the second training, in the latent space, the fourth embedding vector encoded by the second encoder 230 from genotype and responsiveness condition information may be located in a space that reflects (or estimates) compound information corresponding to a drug suitable for the corresponding genotype and responsiveness condition.

In step S130, the system 10 performs third training for the diffusion model 240.

The diffusion model 240 is a conditional diffusion model 240, which generates a representation vector V2 generated from random noise according to the directionality of the generation condition (the fourth embedding vector or condition representation vector V1).

The diffusion model 240 is trained to output the representation vector V2 generated from the condition representation vector V1. The generated representation vector V2 is a vector that may be output as compound information by the pre-trained decoder 220.

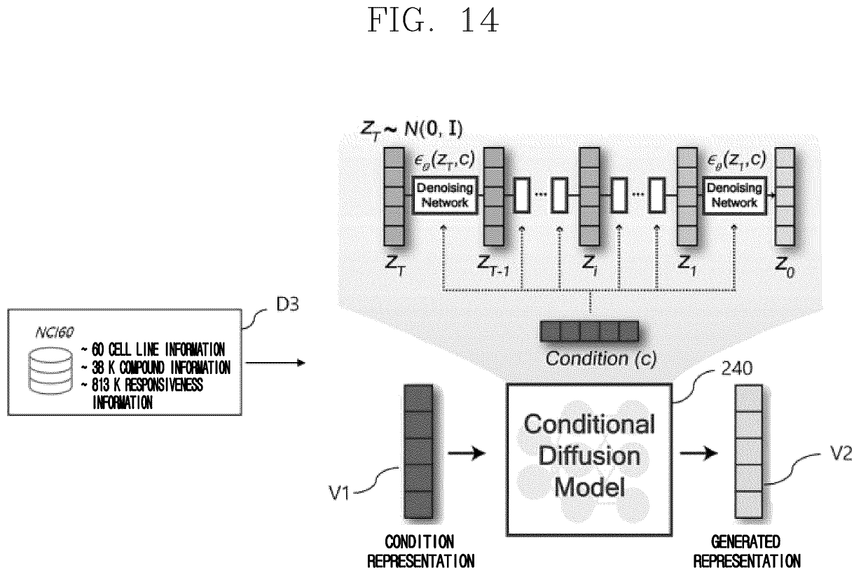

With reference to FIG. 14, the third training data D3 for training the diffusion model 240 includes approximately 60 pieces of cell line information, approximately 38,000 pieces of compound information, and approximately 813,000 pieces of responsiveness information provided from the NCI60 database. When the second training data D2 was a cell line-centric drug response dataset regarding responsiveness information including more diverse cell line information, the third training data D3 may be referred to as a drug-centric drug response dataset regarding responsiveness information including more diverse compound information. Whereas the former focuses on the ability to reflect various genotypes, the latter may focus on the ability to generate various compounds.

In step S200, the system 10 receives a generation condition as input.

The generation condition may be received from the external device 400 via the communication unit 300.

The generation condition may also be input through an input interface that may be provided in the system 10.

The generation condition includes information on genotype and information on responsiveness.

In generating a new compound using a neural network model that has completed training, only very sensitive information may be input as the generation condition. This is because the effectiveness of the drug is generally aimed at good anticancer capability. Accordingly, the responsiveness information may also be used as a default which is not separately input.

In step S300, the second encoder 230 determines an encoding vector corresponding to the generation condition.

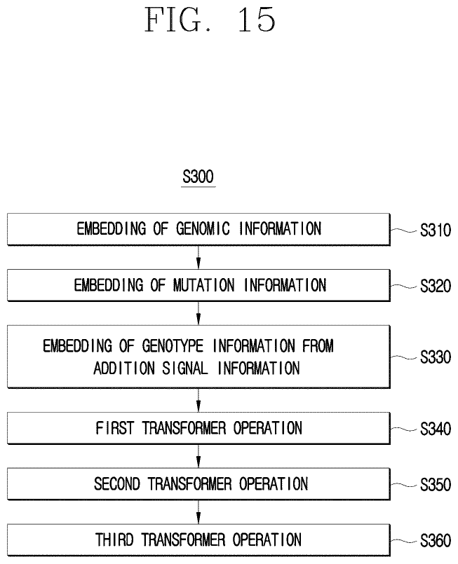

FIG. 15 illustrates step S300 in detail. The description for steps S310 to S360 is the same as the description of steps S1221 to S1226, and is replaced by the above description. However, there is a difference in that the second encoder 230 in steps S1221 to S1226 is not fixed in parameters as training is in progress, whereas the second encoder 230 operating in steps S310 to S360 is fixed in parameters as training is complete.

In step S400, the diffusion model 240 receives, as input, the encoding vector output from the second encoder 230 and determines the generated representation vector.

In step S500, the generated representation vector is decoded by the decoder 220 and determined as compound information.

The compound information output in step S500 may be string information in SMILES format, or structure information regarding an actual compound.

FIG. 16 illustrates a partial stage of the method of generating an anticancer drug candidate compound based on genotype together with related constituent elements according to an embodiment of the present invention.

The components in FIG. 16 may be those for which the first to third training according to step S100 has been completed respectively.

In the leftmost dashed box of FIG. 16, the genotype information (upper part) and the responsiveness information (lower part) in step S200 are represented.

The second dashed box represents step S300. The second encoder 230 receives the genotype information and the responsiveness information as input and outputs an encoding vector related to the generation condition. The output encoding vector is input to the diffusion model 240.

The third dashed box represents step S400. The diffusion model 240 outputs a representation vector generated by sequentially removing random noise based on the encoding vector related to the generation condition.

The fourth dashed box represents step S500. The generated representation vector is input to the decoder 220, converted into compound information, and output.

Further, the system 10 for generating an anticancer drug candidate compound based on genotype according to the present invention may be implemented through a computing device described below, and may perform data processing related to the above-described method of generating an anticancer drug candidate compound based on genotype.

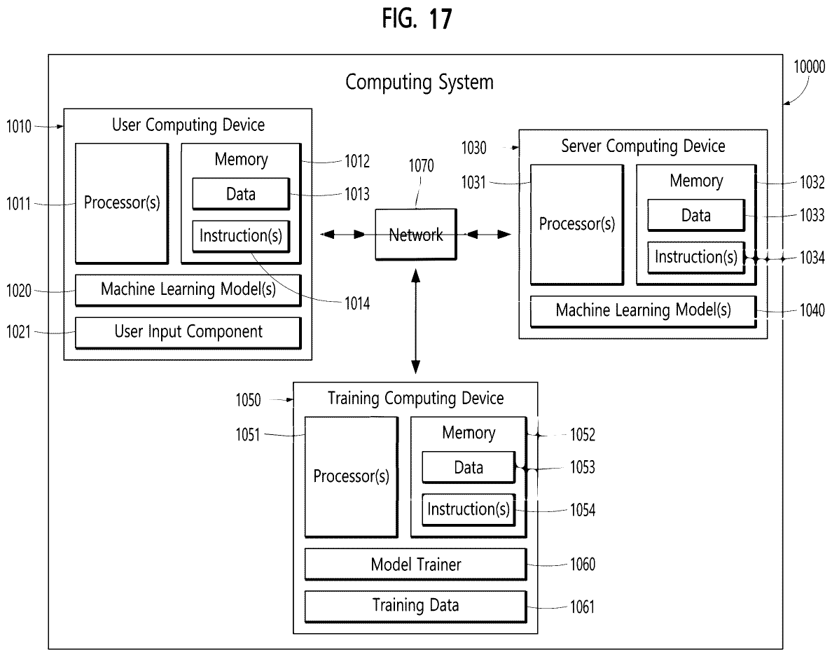

FIG. 17 illustrates an example block diagram of a computing system in which the present invention may be implemented.

Referring to FIG. 17, a computing system (10000) for performing a method for generating anticancer drug candidate compounds based on genotypes according to an embodiment of the present invention may include at least one computing device. In this case, the at least one computing device may be a single-processor or multi-processor computing apparatus.

The components of the at least one computing device of the present invention may include one or more processors, memory, other hardware, and various system components connected (e.g., communicatively, physically, or electrically connected) via a system bus (not shown) that enables data to be transmitted and received among them. The components of the at least one computing device are not limited thereto and may vary widely.

Meanwhile, the at least one computing device included in the computing system (10000) that performs a method for generating anticancer drug candidate compounds based on genotypes may be communicatively connected via a network (1070). For example, the at least one computing device included in the computing system (10000) may be clustered or may be part of a local area network (LAN). Additionally, the at least one computing device may be part of a wide area network (WAN) or connected via at least one of a client-server network or a peer-to-peer network in a cloud environment.

Meanwhile, when the at least one computing device is used in at least one environment among a network environment and a cloud computing environment, the at least one computing device may be connected to at least one of a public network and a private network through a network interface or adapter. In one embodiment, other communication connection devices, such as a modem, may be used to establish communication over the network. The modem may be at least one of an internal modem and an external modem, and may be connected to the system bus through a network interface or a specific mechanism. A wireless network component comprising an interface and an antenna may be coupled to the network through devices such as access points or peer computers. In the present invention, the method by which the at least one computing device is communicatively connected via the network (1070) is not limited thereto and may be implemented by means other than the examples described above.

Furthermore, other computer-type devices and/or systems not illustrated in FIG. 17 may technically interact with the at least one computing device or other systems through one or more connections to the network (1070) via a network interface. Here, the network interface may include network interface equipment such as a physical Network Interface Controller (NIC) or a Virtual Interface (VIF).

The network (1070) of the present invention may include various types of networks such as the Internet, Wireless LAN (WLAN), Wireless Fidelity (Wi-Fi), Wi-Fi Direct, Digital Living Network Alliance (DLNA), Wireless Broadband (WiBro), Worldwide Interoperability for Microwave Access (WiMAX), High Speed Downlink Packet Access (HSDPA), High Speed Uplink Packet Access (HSUPA), Long Term Evolution (LTE), Long Term Evolution-Advanced (LTE-A), 5th Generation Mobile Telecommunication (5G), Bluetooth™, Radio Frequency Identification (RFID), Infrared Data Association (IrDA), Ultra-Wideband (UWB), ZigBee, Near Field Communication (NFC), Wireless Universal Serial Bus (Wireless USB), and the like. In the present invention, data transmission may be performed based on standard communication protocols such as TCP/IP, HTTP, SSL, and others.

The computing system (10000) for performing a method for generating anticancer drug candidate compounds based on genotypes according to the present invention may include at least one of a user computing device (1010), a training computing device (1050), and a server computing device (1030).

The user computing device (1010) according to the present invention may be understood as a computing device including at least one processor (1011) and memory (1012) for performing the method for generating anticancer drug candidate compounds based on genotypes. For example, the user computing device (1010) may include at least one computing device selected from among a smart phone, smart TV, laptop computer, desktop computer, digital broadcasting terminal, personal digital assistant (PDA), portable multimedia player (PMP), navigation device, slate PC, tablet PC, ultrabook, and wearable device (e.g., smartwatch, smart glass, and head-mounted display (HMD)).

The at least one processor (1011) constituting the user computing device (1010) may include one or more general-purpose processors and/or one or more special-purpose processors. For example, the at least one processor (1011) of the user computing device (1010) may include at least one or a combination of electrically connected processors selected from the group consisting of: a Central Processing Unit (CPU), a Graphics Processing Unit (GPU), a Tensor Processing Unit (TPU), a Neural Processing Unit (NPU), an Arithmetic Logic Unit (ALU), a Floating Point Unit (FPU), an Application-Specific Integrated Circuit (ASIC), a digital signal processing device (DSPD), a programmable logic device (PLD), a Field Programmable Gate Array (FPGA), a controller, a microcontroller, a microprocessor, and other electrical units for performing specific functions.

Furthermore, the at least one processor (1011) may be configured to execute computer-readable instructions stored in the memory (1012) and/or other commands described in the present specification.

The memory (1012) constituting the user computing device (1010) according to the present invention may include volatile memory, non-volatile memory, fixed media, removable media, magnetic media, optical media, semiconductor media, and/or other types of physically durable storage media.

For example, the memory (1012) may include one or more non-transitory/transitory computer-readable storage media, or combinations thereof, such as Random Access Memory (RAM), Read Only Memory (ROM), Hard Disk Drive (HDD), Solid State Disk (SSD), Silicon Disk Drive (SDD), Electrically Erasable Programmable Read-Only Memory (EEPROM), Erasable Programmable Read-Only Memory (EPROM), flash memory devices, and magnetic disks. It may also include web storage of a server that performs the memory storage function over the Internet.

The memory (1012) may store data and instructions necessary for the at least one processor (1011) to perform operations of an application for generating anticancer drug candidate compounds based on genotypes.

The user computing device (1010) may include one or more user input components (1021) configured to detect user input. For example, the user input component (1021) may also be referred to as a user interface module. The user input component (1021) may include devices such as a touchscreen, computer mouse, keyboard, keypad, touchpad, trackball, joystick, voice recognition module, or other similar devices. However, the present invention does not limit the types of the user input component (1021).

In this context, the user input component (1021) in the present invention is not necessarily limited to a hardware means but may be understood as a channel through which input is received from a user.

Meanwhile, the “user” in the present invention may also refer to an automated agent, script, playback software, or the like that operates on behalf of one or more human users.

A user may interact with the computing system (10000), which includes at least one computing device, through the user input component (1021) using inputted text, touch, voice, motion, computer vision, gesture, and/or other forms of input/output. For example, the user input component (1021) may include one or more user interface (UI) modalities such as a Command Line Interface (CLI), Graphical User Interface (GUI), Natural User Interface (NUI), voice command interface, and/or other UI representations.

One or more Application Programming Interface (API) calls may be made between the user input component (1021) and the user computing device (1010), based on user input received through a user interface and/or from a network.

Herein, the phrase “based on” may be interpreted to include instances where a particular configuration is used as a foundation, modified from, derived from, influenced by, dependent on, or otherwise originating from such configuration.

In some embodiments, the API call may be configured for a specific API and may be interpreted as, or converted into, an API call configured for a different API. In this context, the API may refer to a defined interface or connection between computers or between computer programs.

In one embodiment, the user computing device (1010) may store one or more machine learning models (1020). For example, the user computing device (1010) may include various machine learning models, such as multiple neural networks (e.g., deep neural networks) for performing a method for generating anticancer drug candidate compounds based on genotypes using generation conditions including genotype information, or other types of machine learning models including nonlinear models and/or linear models or may be configured as a combination thereof.

According to an embodiment of the present invention, the user computing device (1010) may perform a method for generating anticancer drug candidate compounds based on genotypes by using a local and/or external machine learning model (1020). Alternatively, the user computing device (1010) may perform the method for generating anticancer drug candidate compounds based on genotypes by using a machine learning model (1040) provided by a server.

According to another embodiment of the present invention, a server computing device (1030) communicating with the user computing device (1010) may provide information of anticancer drug candidate compounds based on genotypes to the user computing device (1010) via an application and/or a web interface, in response to a user request received through the user computing device (1010).

According to yet another embodiment of the present invention, at least a portion of the user computing device (1010) and the server computing device (1030) may be cooperatively operated to perform a method for generating anticancer drug candidate compounds based on genotypes, thereby providing information of anticancer drug candidate compounds based on genotypes to the user.

According to various embodiments of the present invention, the user computing device (1010) and/or the server computing device (1030) may train the machine learning models (1020, 1040) used in the method for generating anticancer drug candidate compounds based on genotypes through interaction with a training computing device (1050) that is communicatively connected via the network (1070).

In this case, the training computing device (1050) may be a computing system separate from the server computing device (1030). Alternatively, in some embodiments, the training computing device (1050) may be a part of the server computing device (1030) or a part of the user computing device (1010).

Meanwhile, the server computing device (1030) may include at least one processor (1031) and memory (1032). Here, the processor (1031) may include at least one or a combination of electrically connected processors selected from among: a Central Processing Unit (CPU), Graphics Processing Unit (GPU), Tensor Processing Unit (TPU), Neural Processing Unit (NPU), Application-Specific Integrated Circuit (ASIC), Arithmetic Logic Unit (ALU), Floating Point Unit (FPU), digital signal processing devices (DSPDs), programmable logic devices (PLDs), Field Programmable Gate Arrays (FPGAs), controllers, microcontrollers, microprocessors, and/or other electrical units for performing specific functions. For example, the at least one processor (1031) may include circuits and transistors configured to execute instructions from the memory (1032).

The memory (1032) constituting the server computing device (1030) according to the present invention may include volatile memory, non-volatile memory, fixed media, removable media, magnetic media, optical media, semiconductor media, and/or other types of physically durable storage media.

For example, the memory (1032) may include one or more transitory/non-transitory computer-readable storage media, or combinations thereof, such as Random Access Memory (RAM), Read Only Memory (ROM), Hard Disk Drive (HDD), Solid State Disk (SSD), Silicon Disk Drive (SDD), Electrically Erasable Programmable Read-Only Memory (EEPROM), Erasable Programmable Read-Only Memory (EPROM), flash memory devices, and magnetic disks. It may also include web storage of a server that performs memory storage functions over the Internet.

Additionally, the server computing device (1030) may further include a data store. For example, the data store may be configured as at least one of a relational database, a NoSQL database, a data warehouse, and a local file system.

The memory (1032) constituting the server computing device (1030) according to the present invention may store data and instructions necessary for the at least one processor (1031) to perform operations of an application for generating anticancer drug candidate compounds based on genotypes.

In one embodiment, the server computing device (1030) may be configured as a single device or as a plurality of computing devices, which may be configured to operate according to a sequential or parallel computing architecture. Additionally, the system may be implemented as a distributed processing system comprising multiple devices connected over a network.

Meanwhile, the training computing device (1050) may include at least one processor (1051) and memory (1052). A model trainer (1060), as a logical component that performs training of at least one machine learning model (1020, 1040), may be implemented in the form of hardware, firmware, or software.

For example, the model trainer (1060) may load training data (1061) stored in a storage device into the memory (1052), and then be executed by the processor (1051). The model trainer (1060) may be configured to perform one or more operations—such as model training, model reconstruction, model validation, and model testing—on at least one machine learning model.

The machine learning model according to the present invention may include at least one of the following: a statistical model, an algorithm, a neural network (NN), a convolutional neural network (CNN), a generative neural network (GNN), a Word2Vec model, a Bag of Words model, a Term Frequency-Inverse Document Frequency (TF-IDF) model, a Generative Pre-trained Transformer (GPT) model (or other autoregressive models), a Proximal Policy Optimization (PPO) model, a nearest neighbor model (e.g., k-nearest neighbor model), a linear regression model, a k-means clustering model, a Q-learning model, a Temporal Difference (TD) model, a Deep Adversarial Network model, and any other type of model described in the present specification.

Specifically, the model trainer (1060) may perform operations for training a machine learning model, and the operations may include at least one of adding, removing, and modifying model parameters. In this case, the training of the machine learning model may be at least one of supervised learning, semi-supervised learning, and unsupervised learning.

In one embodiment, training of the machine learning model may include a step of repeatedly inputting the training data (1061) based on epochs, and iteratively performing the machine learning model training process configured in this manner. Here, an epoch may refer to a unit representing one complete forward and backward pass of the entire training data (1061) set.

In some implementations, different learning methods (e.g., supervised learning, semi-supervised learning, and unsupervised learning) may be applied at different epochs.

The training data (1061) of the present invention may include input data and/or data previously output from at least one machine learning model (e.g., recursive learning feedback).

The parameters of the at least one machine learning model may include at least one of a seed value, model nodes, model layers, algorithms, functions, connections between different machine learning models, connections between parameters, constraints of the machine learning model, and other digital components that influence the output of the machine learning model.

In this case, a model connection between different machine learning models may include or represent relationships between model parameters and/or between models, which may be dependent, interdependent, hierarchical, and/or static or dynamic.

The combination and configuration of the model parameters described herein may be too complex to be maintained or utilized by human cognitive capabilities.

The present invention does not limit the parameters of machine learning models to those described in the embodiments, and a single machine learning model may include a plurality of model parameters.

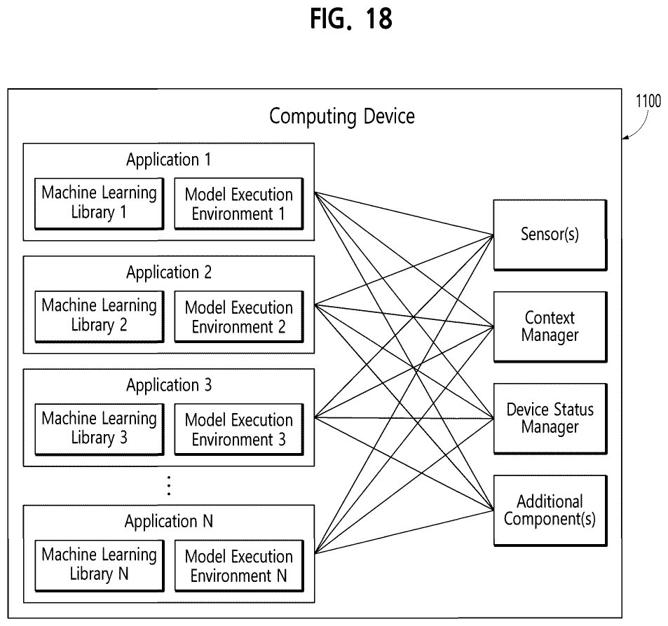

Meanwhile, FIG. 18 illustrates an example block diagram of a computing device (1100), which may be included in the user computing device (1010), the server computing device (1030), or the training computing device (1050), as one embodiment of the computing system (10000) in which the present invention may be implemented.

As shown in FIG. 18, the computing device (1100) may include at least one application (e.g., Application 1 to Application N), and each of the at least one application may include a machine learning library and a model execution environment for performing a method for generating anticancer drug candidate compounds based on genotypes using machine learning.

Each of the at least one application included in the computing device (1100) may communicate via an Application Programming Interface (API) with one or more components within the computing device (1100), such as sensors, a context manager, a device state manager, or additional components.

In one embodiment, the at least one application may interface with device components by, for example, receiving sensor data or state data via a public or dedicated API, or transmitting prediction results to an output device.

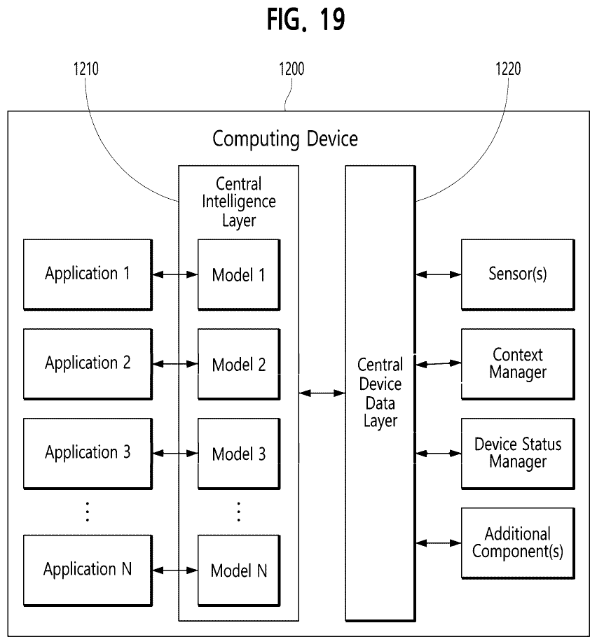

Meanwhile, FIG. 19 illustrates an example block diagram of a computing device (1200), which is one component of the computing system (10000) performing the method for generating anticancer drug candidate compounds based on genotypes according to an embodiment of the present invention, from another perspective.

The computing device (1200) according to the present invention may include at least one application (e.g., Application 1 to Application N), and each of the at least one application may communicate with a central intelligence layer (1210). Each application may interact with a shared model within the central intelligence layer (1210) via an API (e.g., a common API).

The central intelligence layer (1210) may include one or more machine learning models and may either share them among multiple applications or provide them independently to each application. In one embodiment, the central intelligence layer (1210) may be integrated as part of the operating system or implemented as a separate logical layer.

Additionally, the central intelligence layer (1210) may communicate with a central device data layer (1220). The central device data layer (1220) may integratively store generation conditions including genotype information and the like, which are stored within the computing device (1200) and provide them as input data required for generating anticancer drug candidate compounds based on genotypes. Each device component (e.g., sensors, state managers, etc.) may communicate with the central device data layer (1220) via a private API or the like.

The technology described in the present specification may be implemented using a single computing device or multiple computing devices. A machine learning model for performing a method for generating anticancer drug candidate compounds based on genotypes may be executed sequentially or in parallel on a single component or across multiple distributed components. The data store, machine learning models, and applications may be distributed and operated locally or over a network, and these components may be flexibly applied to various system architectures.

The above has described the implementation of the system 10 for generating an anticancer drug candidate compound based on genotype of the present invention as a computing system, but the present invention is not limited thereto. For example, the functionality of the neural network and/or computing device may be distributed among a plurality of computing clusters.

Meanwhile, the present invention described above may be executed by one or more processes on a computer and implemented as a program that may be stored on a computer-readable medium (or recording medium).

Further, the present invention described above may be implemented as computer-readable code or instructions on a medium in which a program is recorded. That is, the present invention may be provided in the form of a program.

Meanwhile, the computer-readable medium includes all kinds of recording devices for storing data readable by a computer system. Examples of computer-readable media include hard disk drives (HDDs), solid state disks (SSDs), silicon disk drives (SDDs), ROMs, RAMs, CD-ROMs, magnetic tapes, floppy discs, and optical data storage devices.

Further, the computer-readable medium may be a server or cloud storage that includes storage and that the electronic device is accessible through communication. In this case, the computer may download the program according to the present invention from the server or cloud storage, through wired or wireless communication.

Further, in the present invention, the computer described above is an electronic device equipped with a processor, that is, a central processing unit (CPU), and is not particularly limited to any type.

Meanwhile, it should be appreciated that the detailed description is interpreted as being illustrative in every sense, not restrictive. The scope of the present invention should be determined on the basis of the reasonable interpretation of the appended claims, and all of the modifications within the equivalent scope of the present invention belong to the scope of the present invention.

The terminology used herein is used for the purpose of describing particular embodiments only and is not intended to limit the present invention. The terms “comprises,” “comprising,” “includes,” “including,” “containing,” “has,” “having” or other variations thereof are inclusive and therefore specify the presence of stated features, integers, steps, operations, elements, and/or components, but do not preclude the presence or addition of one or more other features, integers, steps, operations, elements, components, and/or groups thereof.

It should be noted that, even if constituent elements are substantially identical, ordinal numbers used in claims and ordinal numbers used in the description of the invention may differ depending on the order of presentation.

### DESCRIPTION OF REFERENCE NUMERALS

10: System
100: Processor
200: Memory
210: First encoder
220: Decoder
230: Second encoder
231: First module
2311: Gene embedding block
2312: Mutation signal generation block
2313: Addition signal generation block
232: Second module
2321: First transformer block
2322: Second transformer block
2323: Third transformer block
240: Diffusion model
300: Communication unit
400: External device

What is claimed is:

1. A system for generating an anticancer drug candidate compound based on genotype, the system comprising:
a processor configured to determine compound information corresponding to a generation condition including genotype information; and
a memory,
wherein the memory includes:
a first encoder configured to determine an encoding vector corresponding to the generation condition;
a diffusion model configured to determine a representation vector generated from the encoding vector;
a decoder configured to determine the compound information by decoding the generated representation vector, and
wherein the diffusion model is a conditional diffusion model configured to determine the generated representation vector by removing noise from random noise based on the encoding vector.

2. The system of claim 1, further comprising:
a second encoder trained with parameters associated with the decoder that encodes a compound to determine an embedding vector,
wherein the first encoder is trained such that a distance relationship between the encoding vector and the embedding vector by the second encoder is trained,
wherein the training is performed using pair information of already known genotypes and drugs known to be responsive to the corresponding genotypes as training data, and
wherein the training method is performed by contrastive learning.

3. The system of claim 1, wherein the first encoder comprises:
a first module configured to generate an embedding vector from the genotype information; and
a second module configured to generate the encoding vector corresponding to the generation condition by performing transformer operation on the embedding vector.

4. The system of claim 3, wherein the second module includes a plurality of transformer blocks configured to compute association relationships for different genotypes, and
wherein a first transformer block among the plurality of transformer blocks is configured such that the association computation is limited to computation for adjacent genes.

5. The system of claim 2, wherein the genotype includes partial genomic information of entire human genomic information, and
wherein the partial genomic information is selected from among genomic information related to clinically known cancers.

6. The system according to claim 1, wherein the genotype includes partial genomic information of entire human genomic information, and
wherein each genomic information is associated with mutation presence information, the mutation presence information including base sequence mutation (MUT, Mutation), copy number amplification (CNA), and copy number deletion (CND).

7. A method of generating an anticancer drug candidate compound based on genotype, performed by at least one processor of a computing device, the method comprising:
inputting a generation condition including genotype information;
determining an encoding vector corresponding to the generation condition;
determining a representation vector generated from the encoding vector; and
determining compound information by decoding the generated representation vector,
wherein the generated representation vector is generated by a conditional diffusion model that removes noise from random noise based on the encoding vector.

8. The method of claim 7, further comprising:
using a second encoder trained with parameters associated with the decoder to encode a compound and determine an embedding vector,
wherein the encoding vector determined from the generation condition is trained such that a distance relationship between the encoding vector and the embedding vector determined by the second encoder is learned,
wherein the training is performed using pair information of already known genotypes and drugs known to be responsive to the corresponding genotypes as training data, and
wherein the training method is performed by contrastive learning.

9. The method of claim 7, wherein determining the encoding vector comprises:
generating, by a first module, an embedding vector from the genotype information; and
generating, by a second module, the encoding vector corresponding to the generation condition by performing transformer operation on the embedding vector.

10. The method of claim 9, wherein the second module includes a plurality of transformer blocks configured to compute association relationships for different genotypes, and wherein a first transformer block among the plurality of transformer blocks is configured such that the association computation is limited to computation for adjacent genes.

11. The method of claim 8, wherein the genotype includes partial genomic information of entire human genomic information, and wherein the partial genomic information is selected from among genomic information related to clinically known cancers.

12. The method of claim 7, wherein the genotype includes partial genomic information of entire human genomic information, and wherein each genomic information is associated with mutation presence information, the mutation presence information including base sequence mutation (MUT, Mutation), copy number amplification (CNA), and copy number deletion (CND).

13. A program stored in a non-transitory computer-readable storage medium, executed by one or more processes in an electronic device, wherein the program includes instructions to perform:
inputting a generation condition including genotype information;
determining an encoding vector corresponding to the generation condition;
determining a representation vector generated from the encoding vector; and
determining compound information by decoding the generated representation vector,
wherein the generated representation vector is generated by a conditional diffusion model that removes noise from random noise based on the encoding vector.

14. The non-transitory computer-readable storage medium of claim 13,
wherein the instructions, when executed by one or more processors, cause the one or more processors to use a second encoder trained with parameters associated with the decoder to encode a compound and determine an embedding vector,
wherein the encoding vector determined from the generation condition is trained such that a distance relationship between the encoding vector and the embedding vector determined by the second encoder is learned,
wherein the training is performed using pair information of already known genotypes and drugs known to be responsive to the corresponding genotypes as training data, and
wherein the training method is performed by contrastive learning.

15. The non-transitory computer-readable storage medium of claim 13,
wherein the instructions, when executed by one or more processors, cause the one or more processors to determine the encoding vector by:
generating, by a first module, an embedding vector from the genotype information; and
generating, by a second module, the encoding vector corresponding to the generation condition by performing transformer operation on the embedding vector.

16. The non-transitory computer-readable storage medium of claim 15,
wherein the instructions, when executed by one or more processors, cause the one or more processors to utilize the second module including a plurality of transformer blocks configured to compute association relationships for different genotypes, and wherein a first transformer block among the plurality of transformer blocks is configured such that the association computation is limited to computation for adjacent genes.

17. The non-transitory computer-readable storage medium of claim 14,
wherein the instructions, when executed by one or more processors, cause the one or more processors to use genotype information including partial genomic information of entire human genomic information, and wherein the partial genomic information is selected from among genomic information related to clinically known cancers.

18. The non-transitory computer-readable storage medium of claim 13,
wherein the instructions, when executed by one or more processors, cause the one or more processors to use genotype information including partial genomic information of entire human genomic information, and wherein each genomic information is associated with mutation presence information, the mutation presence information including base sequence mutation (MUT, Mutation), copy number amplification (CNA), and copy number deletion (CND).
